(19) United States
(12) Patent Application Publication
(10) Pub. No.: US 2016/0120501 A1
(43) Pub. Date: May 5, 2016

(54) ULTRASONIC SIGNAL PROCESSING FOR BONE SONOGRAPHY

(71) Applicant: THE GOVERNING COUNCIL OF THE UNIVERSITY OF TORONTO, Toronto (CA)

(72) Inventors: Amir MANBACHI, Toronto (CA); Richard S.C. COBBOLD, Toronto (CA)

(21) Appl. No.: 14/893,647

(22) PCT Filed: May 23, 2014
(86) PCT No.: PCT/CA2014/050485

§ 371 Date: Nov. 24, 2015

Related U.S. Application Data
(60) Provisional application No. 61/827,284, filed on May 24, 2013.
(60) Provisional application No. 61/827,276, filed on May 24, 2013.

(51) Int. Cl. A61B 8/08; A61B 8/12; A61B 17/17; A61B 8/00; A61B 17/16

(52) U.S. Cl. CPC A61B 8/0875; A61B 8/5207; A61B 8/4494; A61B 8/523; A61B 17/1671; A61B 17/1703; A61B 8/5269; A61B 8/12; A61B 8/461

(57) ABSTRACT

This invention relates to ultrasound signal processing methods and systems for use in bone imaging and orthopedic surgery. Signal processing methods and systems for low-frequency bone image guidance, particularly during spinal fusion surgery and the process of pedicle screw insertion are provided.

### CROSS REFERENCE TO PRIOR APPLICATIONS

This application claims priority under the Paris Convention from U.S. Application No. 61/827,276, filed on May 24, 2013 and U.S. Application No. 61/827,284, filed on May 24, 2013, the entire contents of which are incorporated herein by reference.

### FIELD OF THE INVENTION

The present invention relates to methods and systems for processing ultrasound signals. More particularly, the present invention relates to ultrasonic signal processing related to ultrasonic bone sonography, including imaging for orthopedic surgery.

### BACKGROUND OF THE INVENTION

Ultrasound imaging has found extensive and growing applications in diagnostic medicine in the past few decades including diagnostic imaging of bone.

In the process of ultrasound imaging, ultrasound signals are transmitted to a target, reflected from the target and received, and the received signal echoes are then processed to form an ultrasound image.

To transmit ultrasound signals, an ultrasound imaging apparatus generally includes a transducer or a transducer array and a pulser for driving the transducer(s). Each transducer generates ultrasound signals in response to the pulse applied from the pulser. During transmission of the ultrasound signal in arrays, a timing point of the ultrasound generation at each transducer is controlled, thereby transmit-focusing the ultrasound signals at a predetermined position within the target. Ultrasound signals reflected from the target are received by the transducer (array). The power of the received signals is decreased when the signal is passed through a dense medium, such as human tissue. As a result, when the target is located deep in the body, the desired information is challenging to obtain by ultrasound imaging. That is, the received signals have high noise disturbances and thus it is difficult to decipher the image from the reflected ultrasound signals. Accordingly, there is desire for techniques that can transmit increased energy to imaging media without exceeding the amount of power administered to a subject deemed permissible by various regulatory authorities.

Increasing the frequency of the ultrasound pulse may enhance the resolution of imaging. For example, IntraVascular UltraSound (IVUS), used in cardiovascular diagnostics, is possible due to transmitting signals through soft tissue at relatively high frequencies (i.e., >20 MHz). However higher frequencies are susceptible to loss of signal penetration depth due to the higher attenuation of the ultrasound signals for higher frequency components.

In particular, ultrasound imaging of bone, particularly cancellous (trabecular) bone, has proved more challenging due to higher acoustic attenuation. Cancellous bone has a complex structure consisting of a matrix of connected plates and rods, called trabeculae. These spongy structures are interspersed with marrow. The trabeculae are not arranged uniformly, but tend to align in accordance with the stress distribution in the bone. This inhomogeneous, anisotropic, dense composition makes it very difficult to predict and interpret the propagation of acoustic waves in bones. Indeed, trabecular bone has a far higher attenuation, relative to softer more uniform vascular tissue, over the same frequency range, which causes the returned signal to be lost in the background noise. Any decrease in frequency leads to loss in resolution (e.g. signal spatial resolution) and is limited in depth penetration, thereby being disadvantageous and undesirable.

In other high frequency techniques, the reflection of the ultrasound beam from the inner wall of the trabecular bone was near total. This prevents the ultrasound beam from penetrating any significant distance into the trabecular bone, so that the cortical wall could not be imaged.

One method for increasing low frequency ultrasonic signal to noise ratio (SNR), while preserving resolution is coded-excitation.

Coded-excitation techniques were developed in radar technology in an effort to increase radar signal while employing limited transmission power. Pulse compression of the coded waveform enables an increase in the signal-noise-ratio (SNR) while preserving axial resolution. In these methods the duration of the transmitted waveform is substantially increased thereby increasing the total transmitted energy but without increasing the peak transmitted power. As illustrated in FIG. 1 (e.g. FIGS. 1a and 1b), the transmitted waveform has a coded form that could consist of a binary code or a FM (Frequency Modulated) chirp similar to that used by bats in locating objects. Making use of the fact that the exact form of the transmitted signal is known, correlation methods provide a means for extracting spatial scattering information from the received signal, without suffering the loss of resolution associated with a long duration transmitted pulse. The detection and decoding process involves compressing the original transmitted waveform into a signal with a much shorter duration and one that has a similar bandwidth to the transmitted signal (FIG. 2 shown as FIGS. 2A and 2B). The resulting increase in the time-bandwidth product is a direct measure of the SNR (Signal to Noise Ratio) improvement that can be realized. In the case of a radar system, the improvement can be a factor of several thousand, but for ultrasound systems the potential improvement is much more modest.

Coded-excitation techniques have not been fully-explored in medical imaging. However, use of coded-excitation systems and pulse compression techniques for improving the penetration of real-time imaging systems has been considered and it has been suggested that coded-excitation might improve signal-to-noise ratios up to 20 dB, especially in deep tissue regions (U.S. Pat. Nos. 5,984,869 and 6,048,315).

Many different coded waveforms have been devised, the most efficient being frequency modulated chirps and Golay sequences.

Frequency or Chirp modulation, commonly used by bats in their sonar vocalizations, is the use of timing and frequency sweep (i.e., either increasing or decreasing frequency within a range in a given time) (U.S. Pat. No. 2,678,997) (an example illustrated in FIG. 3). The range of frequencies used in chirp modulation allows for both great depth of signal penetration, induced by relatively low frequencies, and improved image quality as a result of also employing higher frequencies. As a result, chirp signaling can increase the quality of received signals obtained in the field of radar or acoustics and improve the signal-to-noise ratio as well as the spatial resolution of an associated image at various signal penetrations. For example, to focus different frequencies in a sonic wave at different depths in an object, a chirped or frequency modulated sonic wave can be passed through a dispersive lens whose focal length varies with frequency (U.S. Pat. No. 3,815,409). The reflected energy can then be applied to a band pass filter to respond to the particular depth being received.

Efforts have been made to apply the ideas developed for chirp radar for ultrasound tissue imaging. However, in ultrasound diagnostic systems because the time-bandwidth product is around two orders of magnitude less than that for radar, the potential improvements in system performance are far less dramatic. One advantage of chirp-coded excitation is that only a single transmission at a time is required with the ultrasound transducer. However, one challenge associated with Chirp techniques is that this form of continuous modulation results in an autocorrelation function that features a single peak bordered on both sides by range side lobes. These side lobes generally are associated with image degradation; and appropriate weighting in the time or frequency domains are typically conducted in order to reduce these chirp-associated side lobes.

Golay codes (GCs) are biphasic codes made up of 1 and −1 values only (illustrated in FIG. 4A-4C, collectively referred to as FIG. 4). Imagine two binary (0 and 1) sequences A and B are of equal length: if the sum of their autocorrelation functions is zero everywhere except at zero lag, the sequences are said to be a complementary pair. Examples of Golay codes include: {+1,+1}, {−1,+1}: {−1,−1,−1,+1}, {−1,−1,+1,−1}: {+1,−1,+1,+1,−1,+1,+1,+1}, {+1,−1,+1,+1,+1,−1,−1,−1}. The last pair of codes is shown in FIG. 4, together with their autocorrelation function. It can be seen that both autocorrelation functions have a peak value of eight at zero lag and that the range sidelobes are equal and opposite. As a result, the sum of the two auto-correlation functions is a triangular pulse with an amplitude of sixteen, a base width equal to twice the code clock period and no range side lobes.

Longer complementary sequences can be readily constructed from shorter sequences. For example, starting from a sequence with a length of 8, sequences with lengths of 16, 32, 64 and 128 can be readily constructed. In contrast to the chirp method, two transmission signals are required for the Golay code, which relies upon pairs of complementary sequences, and which can be made to have no side lobes about the peak of the autocorrelation function. As described above, the employment of this method results in an autocorrelation function with a peak of 2 N and zero side lobes. To date, some patents have incorporated forms of digital coded excitation and compression in soft tissues which enables significant improvements in penetration depth and SNR to be achieved in clinical imaging. Because in the regions closer to the transducer the SNR that can be achieved using conventional methods is quite adequate; as a result, in the shallower regions the Golay code is not used, while the deeper regions benefit from the coded excitation. Golay coding has the advantage that sidelobes in pulse-compressed output are removed. However, ultrasonic signal processing using GC requires two transmission cycles.

GC and chirp modulation can be used identify fast and slow moving ultrasound waves, which may be associated with variance in bone density.

Golay codes have been used previously in bone ultrasound, however, they have been limited to measurements of the acoustic attenuation.

A solution that addresses one or more of the above presented disadvantages and/or limitations for ultrasound imaging of bone is therefore desired.

### SUMMARY OF THE INVENTION

The present invention broadly relates to ultrasound signal processing methods and systems for use in bone imaging and orthopedic surgery.

In one aspect, there is disclosed a system and method for improving SNR of captured images for bone sonography using modulation techniques selected from one or more of: pulse compression, frequency modulation, chirp modulation, and Golay coding.

In a first aspect of the present invention, a method for producing an image of bone using ultrasound is provided. In some embodiments, the method comprises: a) acquiring ultrasound data by: i) transmitting at least one modulated ultrasound signal at the bone to be imaged, wherein the signals are transmitted at frequencies in the range of 0.5 to 5 MHz, wherein the signals are reflected by features within the bone to produce echoes; ii) measuring the echoes, wherein the measured echoes include echoes reflected from multiple spatial locations within the bone; iii) demodulating the echoes; and b) producing an image of the bone from the received demodulated echoes.

In some embodiments of the first aspect, the modulated signal comprises a chirp frequency sweep. In preferred embodiments, the chirp frequency sweep has a central frequency of 2 to 3 MHz and a bandwidth of one octave.

In some embodiments of the first aspect, the modulated signal comprises Golay coding.

In some embodiments of the first aspect, the at least one transmitted modulated ultrasound signal penetrates the bone to a depth of up to 2 cm. In some embodiments of the first aspect, the at least one transmitted modulated ultrasound signal penetrates the bone to a greater depth relative to an un-modulated ultrasound signal transmitted under identical conditions.

In some embodiments of the first aspect, the at least one modulated ultrasound signal is a plurality of modulated ultrasound signals transmitted outwardly by a plurality of transducer elements arranged in a ring configuration, wherein the echoes are received by the plurality of transducer elements, wherein the plurality of transducer elements are in communication with at least one signal processor, wherein the at least one signal processor modulates and de-modulates the ultrasound, wherein the signal processor is in communication with an imaging processor, and wherein the image produced is a cross-sectional image.

In some embodiments of the first aspect, the outwardly directed modulated ultrasound signals are transmitted by a plurality of transducer elements arranged in a first plurality of ring configurations, wherein the echoes are received by a second plurality of ring configurations, wherein the first and second plurality of ring configurations are arranged adjacent to one another in a cylindrical configuration, each ring forming a row in the cylindrical configuration and wherein the image produced is a cylindrical image.

In some embodiments of the first aspect, the plurality of adjacent rings are mounted to or in or integrated with a tool and wherein the tool is inserted in the object to be imaged.

In some embodiments of the first aspect, the method further comprises ultrasound signals directed forwardly relative to the insertional trajectory of the tool, wherein the forwardly directed ultrasound signals are transmitted from a plurality of the transducer elements and wherein the image produced is complimentary to a conical image, wherein the base of the cone is ahead of the tool along the insertional axis.

In preferred embodiments of the first aspect, the imaged bone is a pedicle bone.

In preferred embodiments of the first aspect, the image is generated in real time.

In some embodiments of the first aspect, the image generated has an increased signal to noise ratio relative to an image generated from un-modulated ultrasound signals transmitted under identical conditions.

In some embodiments of the first aspect, the method further comprises: noise reduction by signal (image) averaging, wherein a plurality of modulated ultrasound signals are transmitted at the bone to be imaged, one at a time, and the measured echoes are averaged.

In a second aspect of the present invention, a system for ultrasound imaging of bone is provided. In some embodiments, the system comprises: a) a signal processor, wherein the signal processor codes at least one ultrasound signal and wherein the signal processor decodes at least one received echo of the at least one coded ultrasound signal; b) an ultrasound transducer in communication with the signal processor, wherein the ultrasound transducer transmits the at least one coded ultrasound signal into the bone to be imaged and wherein the ultrasound transducer receives echoes of the coded ultrasound signal reflected from the bone to be imaged; c) an image processor in communication with the signal processor; and d) an image display in communication with the image processor.

### BRIEF DESCRIPTION OF THE DRAWINGS

The features of the invention will become more apparent in the following detailed description in which reference is made to the appended drawings wherein:

FIG. 1 depicts two types of coded excitation schemes that enable the received pulse to be compressed to a fraction of the transmitted pulse length. (a) Binary encoding scheme: a single-cycle sinusoidal transmitted pulse has been assumed. (b) Linear frequency modulated waveform (chirp), together with a square-wave pseudochirp (shown at the bottom).

FIG. 2 consisting of FIG. 2A and 2B depicts a comparison of conventional and pulse compression systems. (a) Conventional pulse-echo system in which the impulse response is that of the transducer and propagation medium. (b) Pulse compression system in which the output is the cross-correlation of the transmitted and received signal waveforms.

FIG. 3 depicts a simple pulse-echo system using an FM chirp and a matched filter.

FIG. 4 consisting of FIG. 4A-4C, depicts properties of an 8-bit Golay code pair. (a) Binary code and its autocorrelation function. (b) Complementary code and its autocorrelation function. (c) Sum of the autocorrelation functions shown in (a) and (b).

FIG. 5 is a block diagram illustrating exemplary method steps of ultrasound signal processing, in accordance with one embodiment.

FIGS. 6A and B are block diagrams illustrating two methods of ultrasound coded excitation signal processing. FIG. 6A illustrates a matched filter method used to generate Amplitude Scans (A-scans) from chirp modulated ultrasound signals. FIG. 6B illustrates a method used to generate A-scans from Golay code modulated ultrasound signals, in accordance with one embodiment (e.g. implemented by the signal processor module 1608 of FIG. 16).

FIGS. 7A and B are design schematics of an exemplary annular ultrasound transducer device employed for obtaining cross-sectional images at any given time, in accordance with respective embodiments. Ultrasound signals are transmitted and echoes are received by the transducer. FIG. 7A illustrates a transducer without matching layers. FIG. 7B illustrates a transducer with two layers of acoustic matching.

FIG. 8 is a block diagram illustrating exemplary method steps for generating an image of bone using modulated ultrasound signal methods of the present invention, in accordance with one embodiment..

FIG. 9 depicts an example of an annular ultrasound transducer having a plurality of ring-shaped transducer arrays, in accordance with one embodiment.

FIG. 10 is a perspective diagram providing exemplary design specifications of an angular sector arch of the cylindrical ultrasound transducer array, in accordance with one embodiment. As will be understood, the dimensions vary based on variability of pedicle morphology from lumbar to cervical (neck) or thoracic (chest) spine.

FIG. 11 is a pictorial representation of a cylindrical ultrasound transducer array incorporated with a drill bit for simultaneous imaging and pilot hole creation in a pedicle bone structure. The device is shown within the anatomical structure of the target.

FIG. 11A is a diagram of a 32-element ultrasound transducer probe, handle and array, in accordance with one embodiment.

FIG. 12 is a sketch illustrating a cylindrical ultrasound transducer array incorporated with the exterior of a drill bit, in accordance with one embodiment.

FIG. 13 is a pictorial representation showing electronic focusing of ultrasound beams in pedicle cross-sectional imaging, in accordance with one embodiment.

FIG. 13A is a block diagram of a system for providing imaging using an ultrasound transducer array, in accordance with one embodiment.

FIG. 14A is a schematic that illustrates how the ultrasound beam focal spot of the ultrasound transducer array is shifted spatially using a phased array technique of introducing electronic delays to the fire timing of each element, thereby allowing the user of the array to “look ahead” of the array, in accordance with one embodiment.

FIG. 14B depicts the insertion trajectory of a pedicle probe, ultrasound signals directed forwardly to allow the user to look ahead of the tip of the device.

FIG. 15 depicts the anatomy of soft cancellous bone and hard cortical bone encapsulating the cancellous bone within a spinal vertebral body (as shown in the left image) and an ultrasound image generated from a pedicle bone (as shown in the right image) using the device disclosed in U.S. Provisional Patent Application titled “Ultrasonic Array for Bone Sonography”, filed May 24, 2013 by the inventors named herein.

FIG. 16 is a block diagram illustrating a system for generating an image of a bone using modulated ultrasound signal methods of the present invention, in accordance with one embodiment.

FIG. 16A is a block diagram depicting exemplary steps for generating an image of a bone using modulated ultrasound signals, in accordance with one embodiment.

FIG. 17A-C depict pulse echo experimental design set up. FIG. 17A: a 1.5 cm thick human cancellous bone on top of microscopic glass slide. FIG. 17B: a 1.5 cm thick human cancellous bone on top of a cortical bone layer. FIG. 17C: a schematic illustrating pulse echo experimental design. Various signal techniques (Single Sinusoidal pulse, Chirp modulation and Golay Code) were compared for a pulse-echo setting of ±1 Vpp.

FIG. 18A-B depict experimental and modeling results of comparing single sinusoidal pulse, chirp modulation and Golay coded ultrasound signals in imaging cancellous bone.

FIG. 19 depicts the average response of 20 one-cycle sine pulses (2 MHz) of an ultrasound signal.

FIG. 20 depicts the results of chirp modulation of an ultrasound signal with a frequency sweep from 0.5 to 3.5 MHz over 0.25 ms.

FIG. 21 depicts the results of Golay code modulation of an ultrasound signal with 512 bits modulated in 2 MHz.

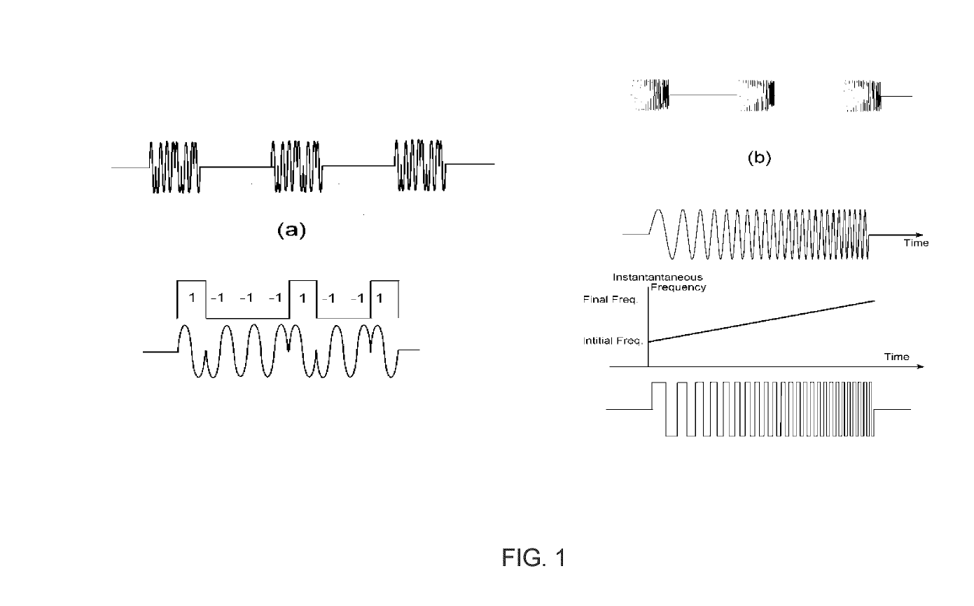

### DETAILED DESCRIPTION OF THE INVENTION

The definitions of certain terms as used in this specification are provided below. Unless defined otherwise, all technical and scientific terms used herein generally have the same meaning as commonly understood by one of ordinary skill in the art to which this invention belongs.

The present invention generally relates to a method for producing an image of bone using ultrasound. It is contemplated that coded-excitation can be used to improve ultrasound signal penetration in bone and increase signal to noise ratio in images generated from ultrasound imaging of bone.

In some embodiments, coded excitation techniques, are particularly useful for imaging bone tissue. Non-coded ultrasound waves go through intense and rapid signal decay in bone due, at least in part, to the heterogeneity of bone tissue. As a result, increased energy must be used to generate enough sensitivity to detect echoes. For example, in a bony tissue such as the pedicle, where target tissue is relatively close, basic pulse echo settings (e.g., a few cycles of sinusoidal excitation) may be ineffective because the echoes could be lost within the transmitted pulse.

In some embodiments, coded excitation techniques, such as chirp frequency sweeping or Golay coding, are advantageous relative to non-coded ultrasound techniques because they allow more energy to be transmitted to a focal depth, for example relative to a one or two cycle pulse, when the same transmission voltage level is applied to a transducer. In some embodiments, this is advantageous because there may be regulatory guidelines regarding the amount of power transmitted into a human body during a medical procedure.

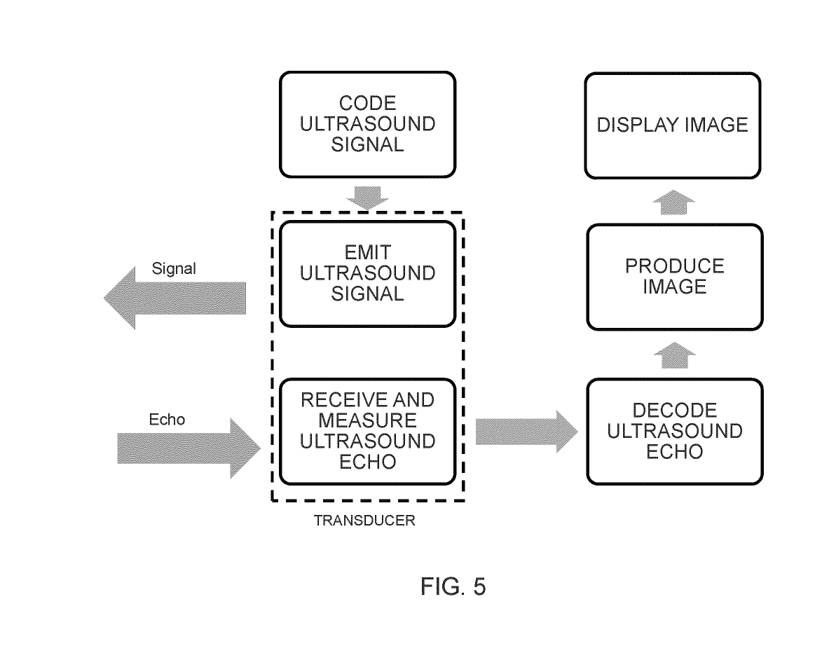

Referring to FIG. 5, in some embodiments of the present invention, the method for producing an image of bone using ultrasound comprises acquiring ultrasound data by generating and transmitting at least one modulated ultrasound signal at the bone to be imaged. In some embodiments, the modulated signals are transmitted at low frequencies in the range of 0.5 to 5 MHz. These relatively low frequencies are preferred for imaging bone, at least because ultrasound imaging within bone results in high signal attenuation, which increases with higher transmit frequencies. The low-frequency modulated signals transmitted into the bone to be imaged are reflected by features within the bone to produce echoes that are measured and then demodulated. The demodulated echoes can then be used to generate an image of the bone.

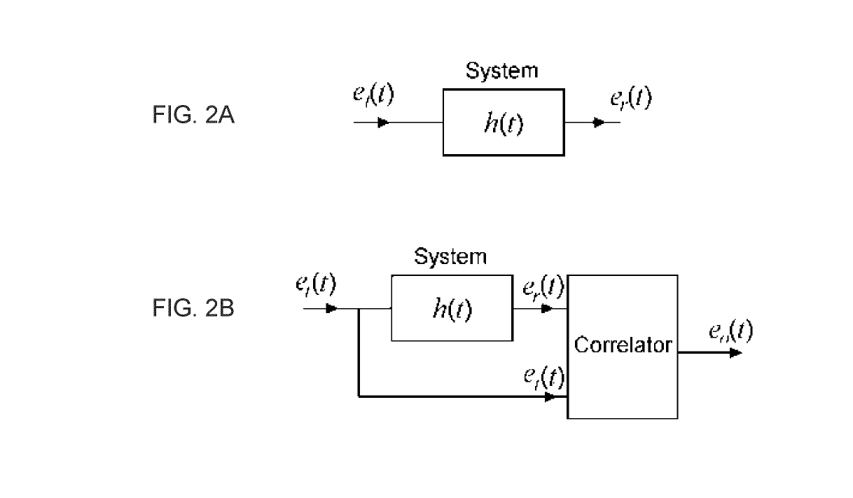

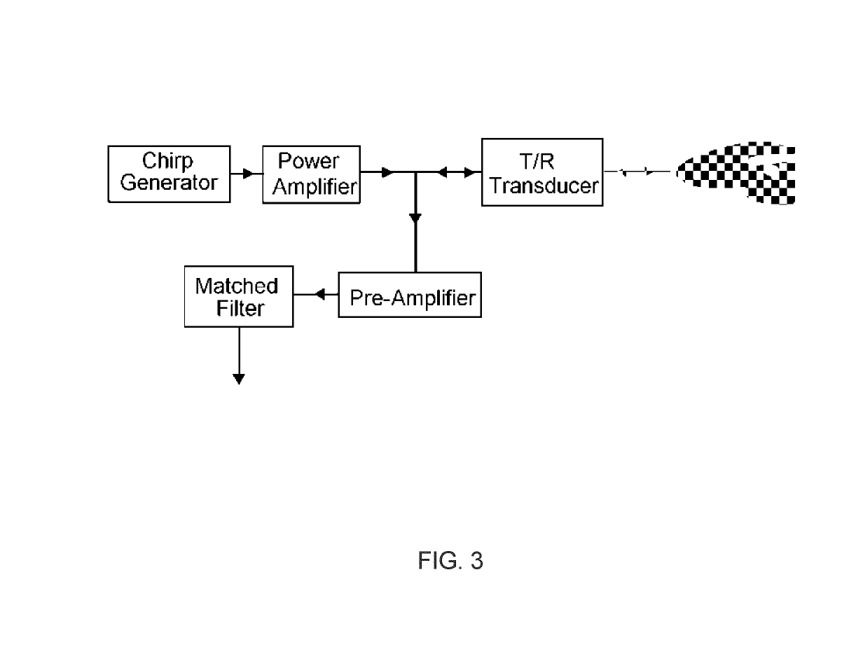

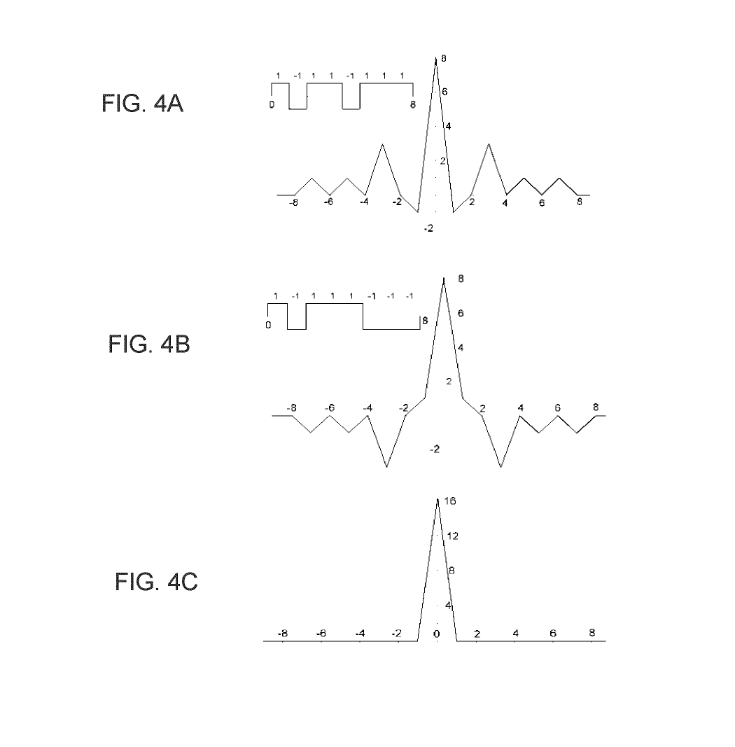

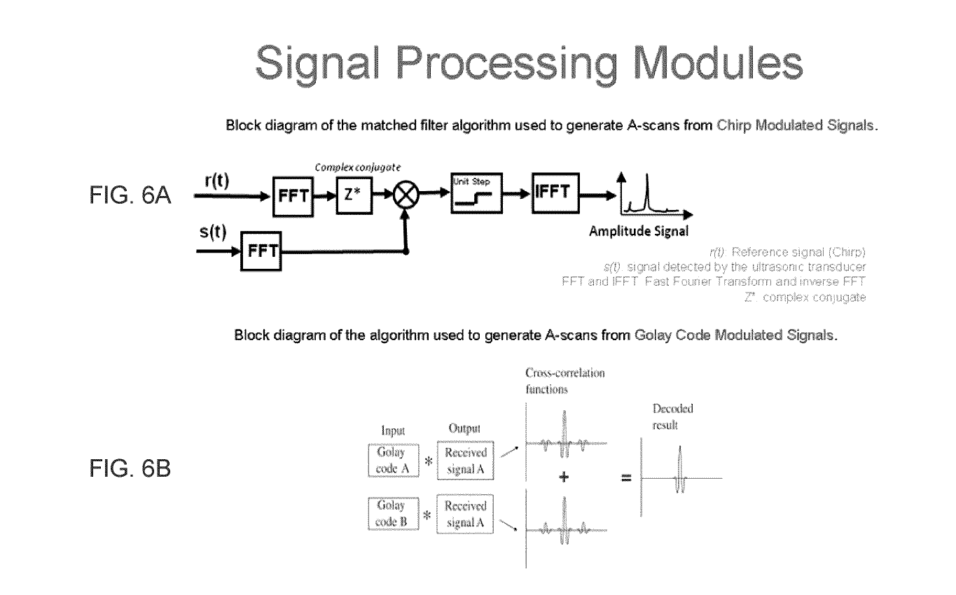

In some embodiments, the modulated signal transmitted into the bone comprises a chirp frequency sweep. Referring to FIG. 2B, the generic steps involved in the pulse compression methods used in Chirp modulation are shown. Referring to FIG. 6A, a matched filter method is used to generate Amplitude Scans (A-scans) from chirp modulated ultrasound signals. In the case of Chirp modulation, the transmitted signal is used as a reference that, when cross-correlated with the received echo, results in an output signal. In preferred embodiments, the chirp frequency sweep has a central frequency of 2 to 3 MHz and a bandwidth of an octave. The low frequencies in the chirp sweep allow for greater depth of signal penetration and the high frequencies in the chirp sweep allow for improved image quality.

In other embodiments, the modulated signal transmitted into the bone comprises Golay coding. Referring to FIG. 6B, an algorithm is used to generate A-scans from Golay code modulated ultrasound signals. In the case of Golay code technique, the steps illustrated in FIG. 2B are carried out and the resultant output signals are summed. Referring to FIG. 6B, each block diagram illustrates processes that are associated with one of the complimentary pairs required for the Golay Code.

In some embodiments, the transmitted modulated ultrasound signal penetrates the bone to a depth of up to 2 cm. In preferred embodiments, the transmitted modulated ultrasound signal penetrates the bone to a greater depth relative to an un-modulated ultrasound signal transmitted under identical conditions.

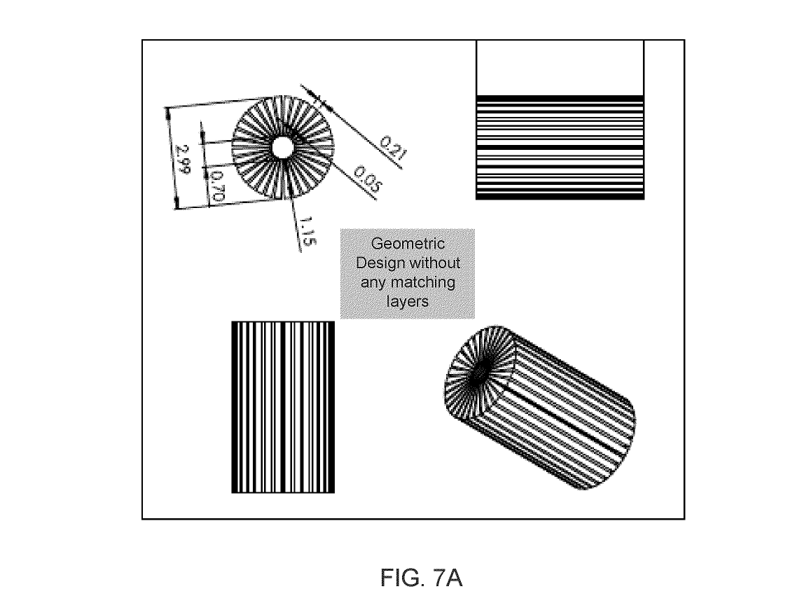

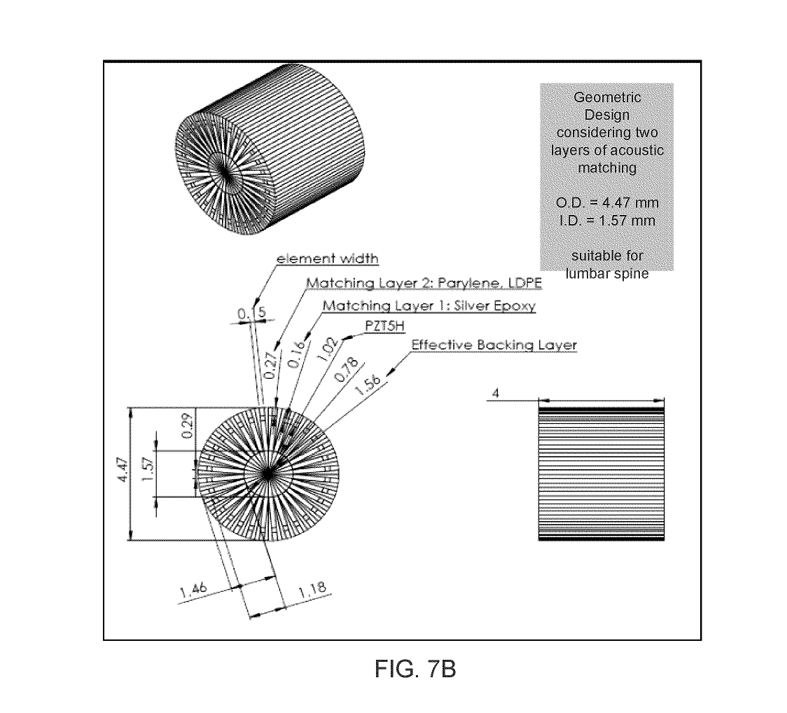

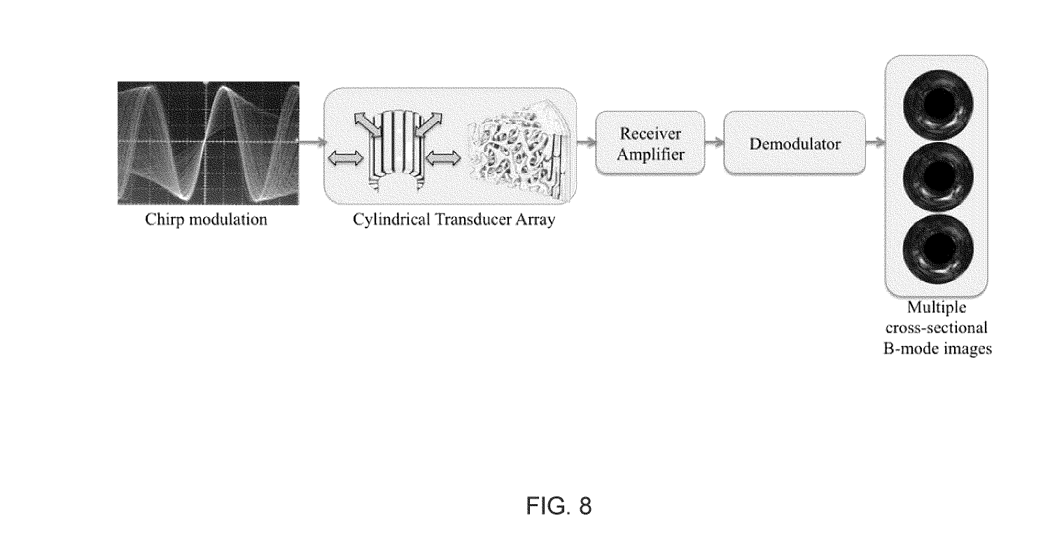

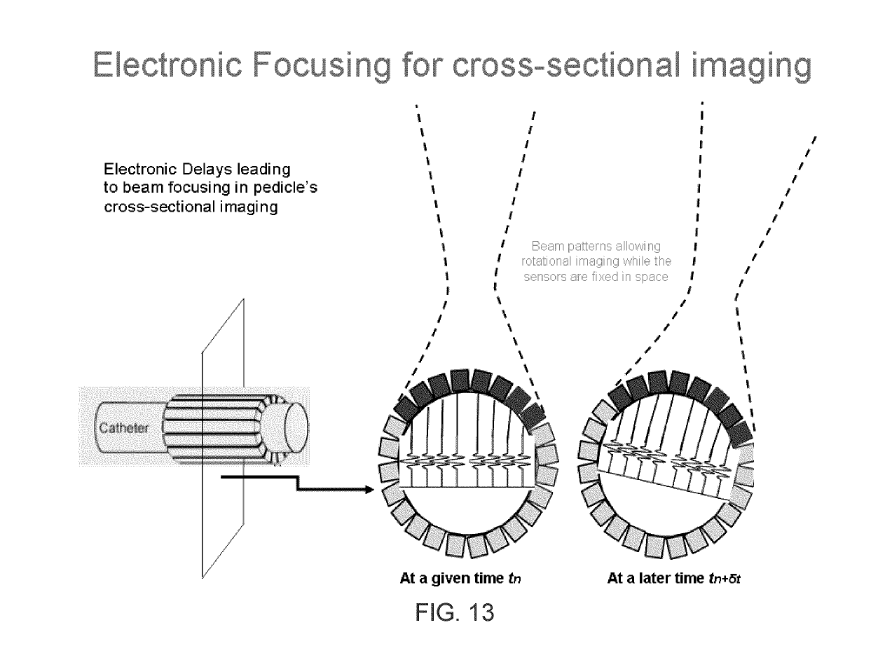

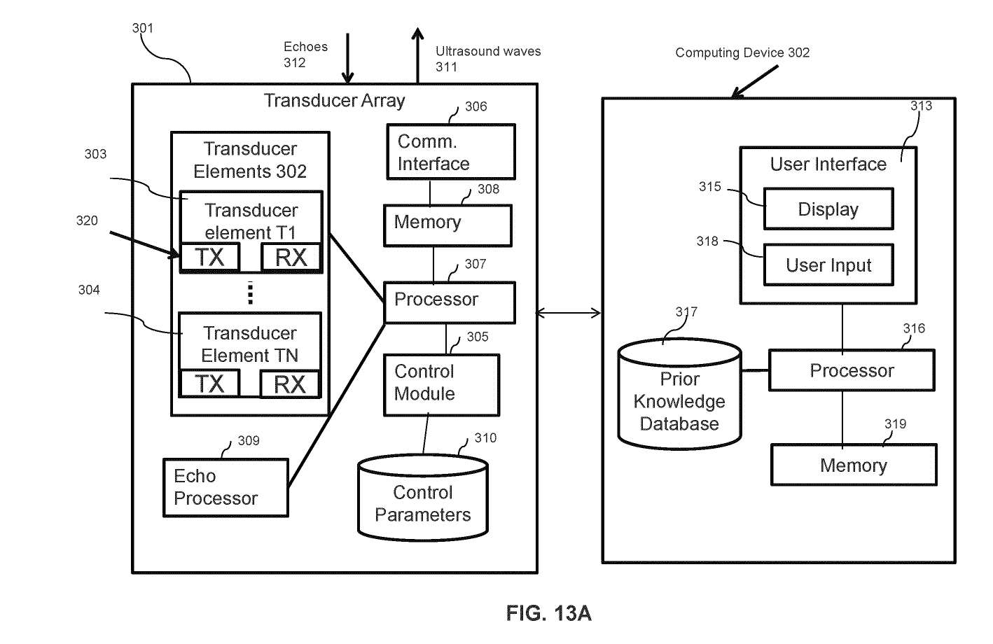

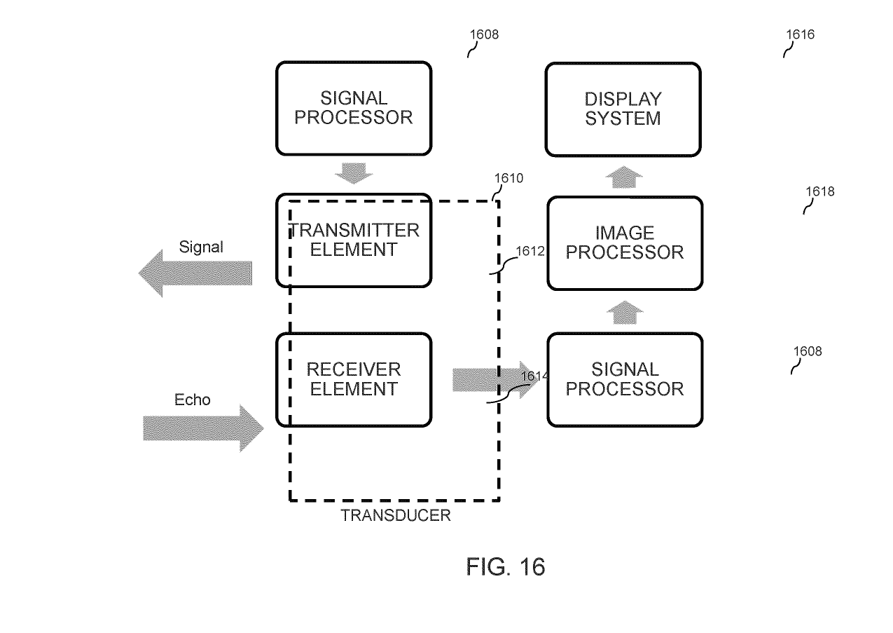

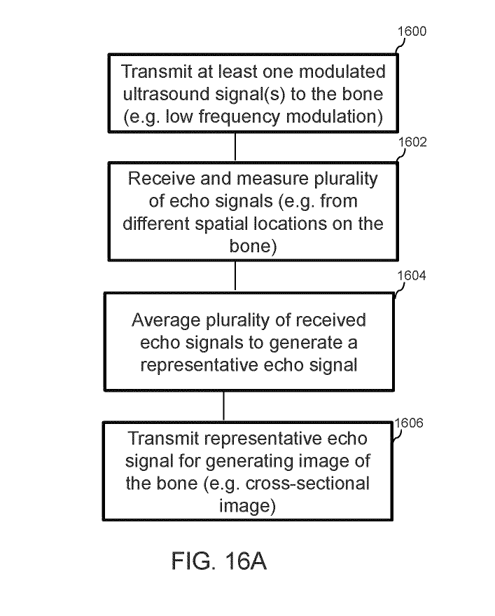

Referring to FIG. 7A, 7B and FIG. 8, in some embodiments, a plurality of modulated ultrasound signals are transmitted outwardly into bone by a plurality of transducer elements arranged in a ring configuration. Echoes of the transmitted signals are received by the plurality of transducer elements, which are in communication with at least one signal processor (e.g. signal processor 1608 in FIG. 16). In some embodiments, the signal processor de-modulates the ultrasound echo signal received. In some embodiments, the signal processor also modulates the transmitted signal (e.g. as discussed with respect to FIG. 16). In other embodiments, separate signal processors are used to modulate and de-modulate the ultrasound signals and echoes, respectively each signal processor in communication with the transducer elements. In some embodiments, the signal processor is in communication with an imaging processor (e.g. as shown in FIG. 16 as image processor 1618 or processor 316 existent on external computing device 302 in FIG. 13A). In some embodiments, the image processor is in communication with an image display (e.g. display 1616 in FIG. 16 or display 315 in FIG. 13A). In some embodiments, the image produced is a cross-sectional image.

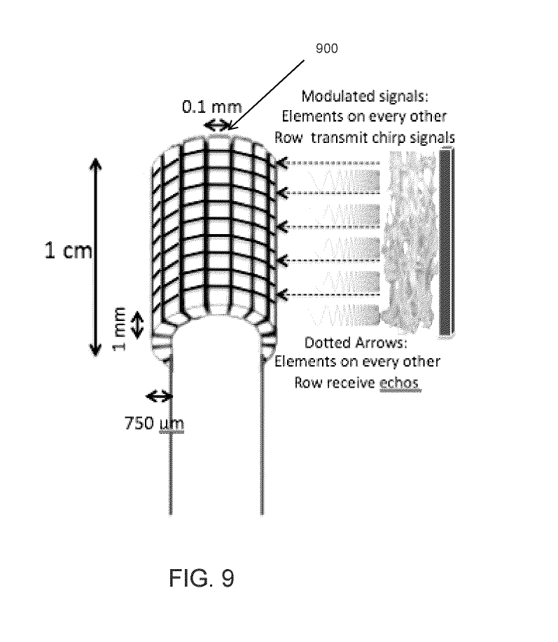

Referring to FIG. 9, there is illustrated a cylindrical ultrasound transducer array 900 for use in generating a three-dimensional image of the pedicle bone's cortical layer. The ultrasound transducer array 900 comprises a plurality of ring-shaped transducer arrays. Dimensions are provided for example purposes, providing proof of principle, particularly for fitting the transducer into the pedicles of lumbar spine. As can be envisaged, dimensions can be varied to suit pedicle morphology differences in lumbar, cervical or thoracic pedicle bones.

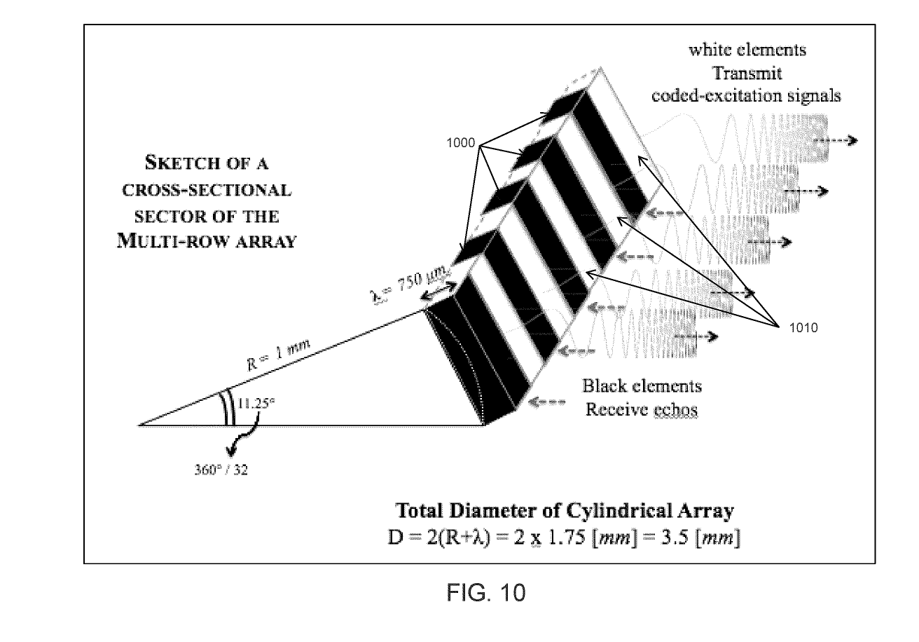

Referring to FIG. 9 and FIG. 10, in some embodiments, the outwardly directed modulated ultrasound signals are transmitted by a plurality of transducer elements arranged in a plurality of ring configurations (e.g. 900). As shown in FIG. 10, a first plurality of transducer elements 10101 arranged in a first plurality of ring configurations are configured for transmitting the outwardly directed modulated ultrasound signals. Referring to FIG. 10, a second plurality of transducer elements 1010 arranged in a second plurality of ring configurations is configured for receiving the echoes generated by the subject (e.g. pedicle bone) in response to interaction with the ultrasound signals. Referring to FIG. 10, the first and second plurality of ring configurations 1010, 1000 are arranged adjacent to one another in a cylindrical configuration, each ring forming a row in the cylindrical configuration and wherein the image produced (e.g. via the image processor 316 of FIG. 13A) is a cylindrical image.

Referring to FIG. 10, as will be understood, the diameters shown are exemplary and are not meant to be limiting in their nature.

In some embodiments the device used to transmit and receive the modulated ultrasound signals and echoes comprises transducer elements where every other row of the plurality of rings of the transducer elements are transmitters and the transducer elements in the rows between the transmitters are receivers. This design is particularly useful when the ultrasound signal is Chirp Modulated, at least because chirp modulated ultrasound signals comprise transmitted pulses that are longer in length than un-modulated signals. Longer pulses allow for a possibility of overlap between transmitted and received signals. As a result, in some embodiments, the transducer array imaging system could employ this alternative row design to overcome potential signal overlap.

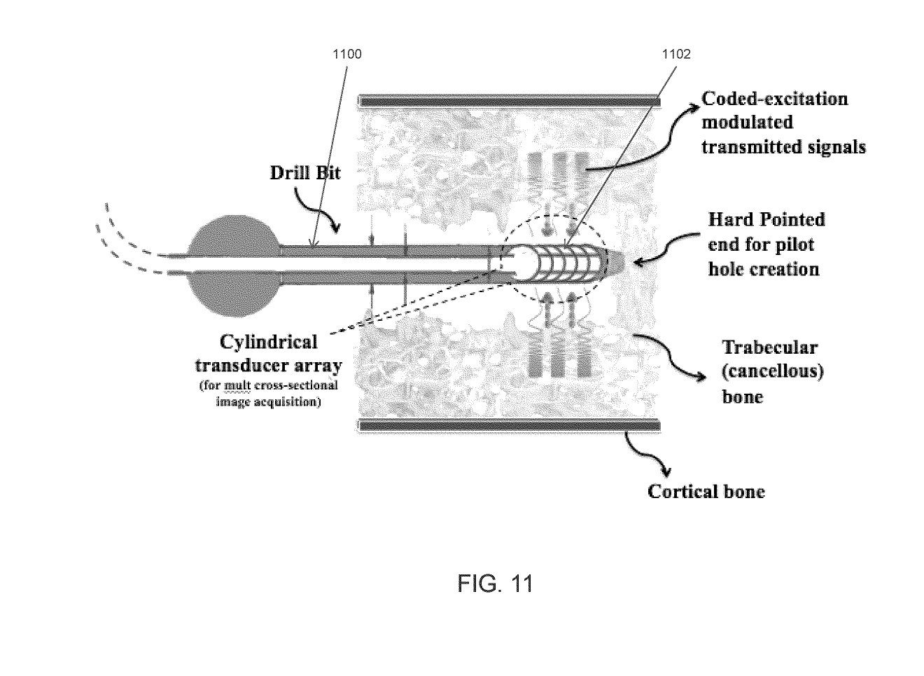

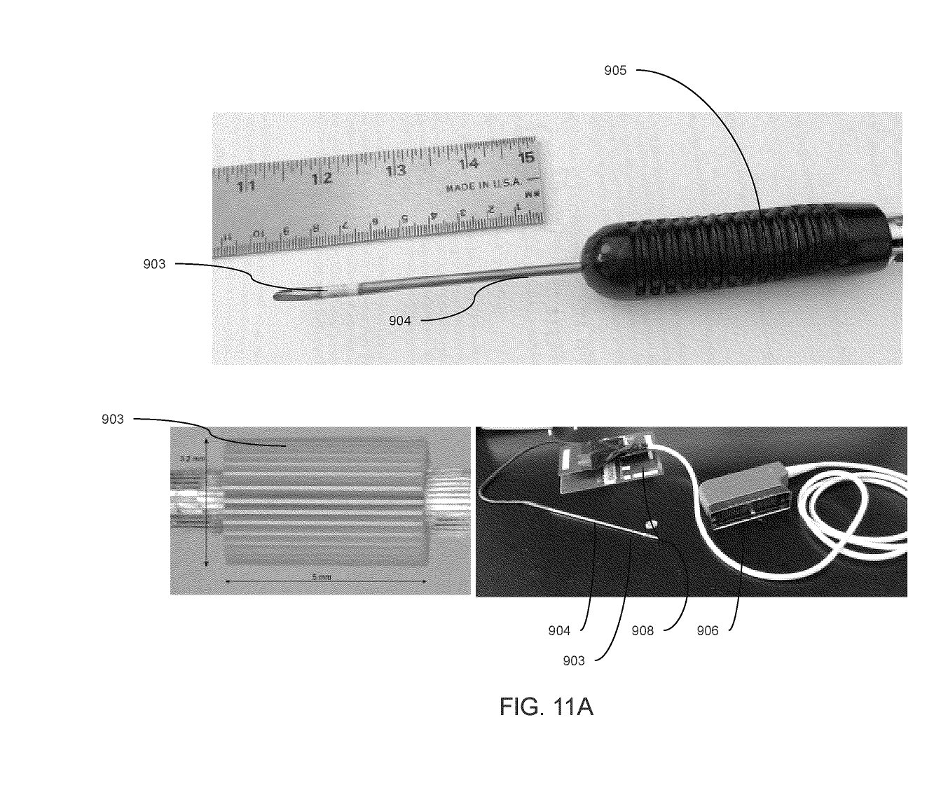

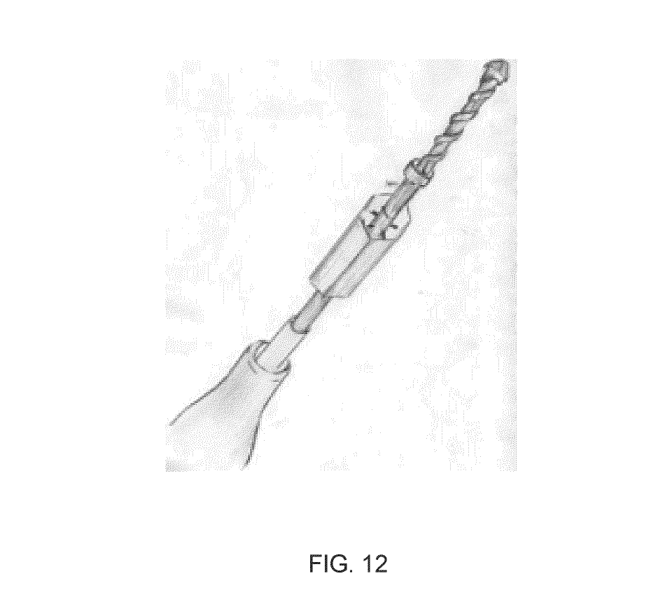

Referring to FIG. 11 and FIG. 12, in some embodiments the plurality of adjacent rings are mounted to or in or integrated with a tool and wherein the tool is inserted in the object to be imaged. Referring to FIG. 11 shown is an embodiment where a cylindrical transducer array 1102 is integrated with a tool bit (e.g. a drill bit 1100) for insertion into a cortical bone.

In other embodiments, the transducer array is integrated with a tool for probing or cannulating bone (e.g. as shown in FIG. 11A). For example, the transducer array of the present invention can be mounted to or integrated with a tool for generating pedicle guide holes or a tool used for pedicle screw placement.

Referring to FIG. 11A, shown is an exemplary device for generating and transmitting modulated ultrasound signals to a subject tissue and for receiving echo signals in response thereto while allowing creation of a hole in the pedicle bone. In this manner, images can be generated in real time while creating the hole in the subject such as pedicle bone. As discussed herein, the echo signals are used for subsequent generation of images representing the subject tissue (e.g. pedicle bone) in accordance with one embodiment. FIG. 11A illustrates the device comprising an ultrasound transducer array, rod and handle in accordance with one embodiment. The device comprises a surgical apparatus having, for example, screwdriver geometry (e.g. tool bit 904), a transducer array 903 is preferably embedded inside an epoxy (to protect it from scratches from bone) and a handle portion 905.

The tool bit 904 is for engaging with a treatment surface (e.g. bone tissue) and for penetration of same for creation of the hole. The transducer array 903 is configured for providing radial imaging from within the target, preferably with a low frequency transducer as described herein to allow for penetration of the tissue while considering signal to noise ratio of the captured image, and preferably having relatively small dimensions. In this manner, FIG. 11A provides a 32 element ultrasound transducer configured for imaging the pedicle bone radially, from within the subject being imaged and without mechanical rotation of the element (e.g. transducer array 903).

In one aspect, the transducer array 903 is employed in a fashion that uses electronic steering rotation in order to obtain cross sectional images (e.g. 360 degree radial imaging) from the pedicle bone. Referring to the bottom right image in FIG. 11A, shown is the transducer array 903 mounted on a stainless steel rod 904 connected to an electrical matching circuitry 908. The electrical matching circuitry 908 is configured for reducing the signal loss as it goes through all the electronic components of the transducer device. A communication interface 906 such as an electrical connector socket is used to interface the hardware to an external computing device such as an ultrasound console. As described herein the transducer array is in communication with a processor for executing instructions according to the embodiments described herein (e.g. processor 307 and/or image processor 316 described in reference to FIG. 13A) for processing the received echo signal (e.g. echo processor 309, processor 307) and generating the corresponding image of the bone (e.g. via image processor 316). As described in FIG. 13A, the processor 307 and/or 316 is configured for executing instructions (e.g. stored on a memory 308 and/or memory 319) for facilitating the measurement and analysis of the echo signals, capturing of images and communicating the reflected echo information (e.g. one or more of control parameters 310 shown in FIG. 13A such as but not limited to: control of timing, delays, direction, electronic focusing, and number of transducer elements engaged at one time for sending the ultrasound waves).). The control parameters 310 have also been referred to as trigger signals herein.

Referring again to FIG. 11A, the transducer array 903 is preferably a 32 element ultrasound imaging array, operating in a low frequency range. The transducer array 903 consists of 32 transducer elements disposed on a cylindrical configuration and embedded within a coating such as an epoxy that protects the elements from scratches from interaction with the bone. In one aspect, the array is configured for being coupled with a tool bit (e.g. any type of screwdriver tip). In one aspect, the tool bit is autoclave-able and sterilize-able.

Referring to FIG. 13, in some embodiments the modulated signals are transmitted and received by a phased annular ultrasound transducer array. Phased array systems pulse and receive signals from the plurality of elements of an array. The plurality of transducer elements is pulsed in a pattern to cause multiple beam components to combine with each other to form a single wave front traveling in the desired direction. Pulsing can occur simultaneously among the transducer elements of the array or at a time delay relative to one another. The plurality of receiver elements combine the received echo input into a single presentation (e.g. single signal for computing the image representation of the echo signals). In one aspect, the manipulation of the echo signals to generate a single presentation is performed by the echo processor 309 (e.g. FIG. 13A). Because phasing method permits electronic beam shaping and steering, it is possible to generate various ultrasonic beam profiles from a single probe assembly.

It is contemplated that in some embodiments, executable instructions stored in a memory 308 coupled to a transducer array 301 for execution by one or more processors 307 (e.g. as shown in FIG. 13A) can be used to control the modulated ultrasound beam angle, focal distance, and beam spot size. These parameters can be dynamically scanned at each inspection point to optimize incident angle and signal-to-noise for each part geometry. The parameters can be stored in a database comprising control parameters 310 for use by a control module 305 in cooperation with the processor 307 to control the operation of one or more transducer elements (e.g. including but not limited to: beam angle, focal distance, beam spot size, selection of one or more transducer elements 302 for generating the signal).

Referring to FIG. 13A, the transducer comprises a transducer array 301. The transducer is annular, cylindrical, or conical in shape depending upon the type of subject being imaged and the desired focus/signal to noise ratio. As described herein, the annular transducer can be configured with or without matching layers. Whether matching layers are used is defined by the size of the transducer device allowed for the pedicle bone (e.g. absence of matching layer allows smaller device), or increased desired sensitivity (e.g. increased sensitivity is provided by matching layers). The parameters for configuring the transducer can be stored in a database (e.g. prior knowledge database 317) on the computing device 302. In this manner, the efficiency of a particular use and images obtained from a transducer are stored in database 317 for defining matching parameters by the processor 316 for subsequent transducer array use.

Referring to FIG. 13A, the transducer array 301 comprises one or more transducer elements (303, 304). Each transducer element 303, 304 further comprises a transmitter and/or a receiver elements 320. The transmitter elements are configured for transmitting the ultrasound waves 311 to a subject (e.g. a pedicle bone) and the receiver elements are configured for receiving the echoes 312. The operation of the transducer elements 302 is controlled by a processor 307 in communication with a control module 305 for triggering the operation of one or more transducer elements 303 in generating the transmitter and/or receiver signals. As will be described, the control module 305 is further in communication with control parameters 310 for defining timing, delay and selection of one or more transducer elements 302. In some embodiments, the transducer array 301 is in communication (e.g. via a communication interface 306) with an external computing device 302 for generating the images from the received echoes 312. The transducer array 301 may be directly electrically coupled to the computing device 302 or may be in wireless communication therewith (e.g. Bluetooth). Referring to FIG. 13A, the annular transducer array 301 is in communication with an imaging processor 316 and an image display 315 for generating images of the pedicle bone.

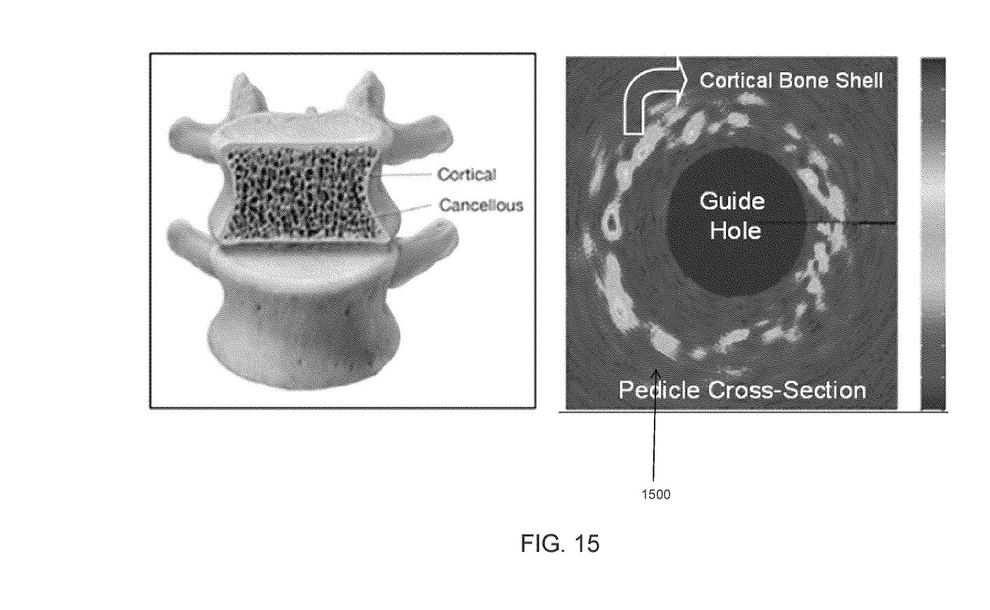

In one embodiment, in response to generating the ultrasound waves 311 to a subject (e.g. a pedicle bone), the subject tissue provides one or more echo signals 312. The transducer array 301 is configured to receive the echo signals 312 and process the echo signals 312 via an echo processor 309. The echo processor 309 is configured to translate the echo signals 312 (e.g. by averaging, defining a specific focal point to provide emphasis to particular echo signals, by ranking the echo signals and providing a weighted gain) to a response signal indicative of the image of the structure. The response signal also referred to as a representative echo signal is provided to the computing device 302 for processing by the processor 316 and generating the image on the display 315. Referring to FIG. 13A, the computing device 302 further comprises a user interface 313 for receiving user input 318 to manipulate the image and/or provide control parameters for affecting the resolution, timing, and/or delay as stored in the control parameters 310. In this manner, cross-sectional images of a hollow structure can be obtained by ultrasound ‘pulse-echo’, which is based on the time that it takes for an excitation pulse to travel within the bone, hit the thick cortical target and return back to the transducer. For example, a cross-sectional image of a pedicle is shown in FIG. 15.

Referring to FIG. 13A, the computing device 302 further comprises a memory 319 for storing instructions thereon and for execution by the processor 316 in generating the image from the echo signals 312 as provided by the echo processor 309.

Referring to FIG. 13A, in some embodiments, the plurality of transducer elements 302 are arranged in a ring-like configuration that allows a circumferential image to be taken in real time (e.g. as provided to an image processor 316 for generating the image on a display 315). The transducer elements 302 are configured to transmit ultrasound signals 311 and/or receive ultrasound echoes 312 for generating the image.

Referring again to FIG. 13A, one advantage of a transducer array 301 having a plurality of transducer elements 302 is that a user (e.g. via the computing device 302) can deliberately trigger the elements one at a time (e.g. trigger the operation of one or more transmitter 320 in one or more transmitter elements 302), in sequence, simultaneously (e.g. multiple transmitter elements 303 and 304) or with delays with respect to the adjacent elements.

Indeed, in some embodiments of the present invention, the transducer array 301 can transmit ultrasound signals 311 from a specified sub-set of transducer elements 302. The sequence of transmitted ultrasound signals 311 could include desired time delays, which might be useful for improving focus of the ultrasound waves (i.e., beams), which could result in improved image resolution and quality. For example, when waves originating from two or more sources interact with one another, phasing effects occur, which lead to an increase or decrease in wave energy at the point of wave combination. When elastic waves of the same frequency meet such that their displacements are synchronized (i.e., in phase or at 0 degree phase angle), the wave energies combine and create a larger amplitude wave. If they meet such that their displacements are opposite (i.e., 180 degrees out of phase), then the wave energies will cancel each other. When elastic waves meet at phase angles between 0 and 180 degrees, a range of intermediate stages between full addition of energy and full cancellation of energy can occur. By varying the timing of the waves from a large number of sources, it is possible to use these effects to both steer and focus the resulting combined wave front. The timing information of the waves for triggering the operation of one or more transducer elements 302 is stored in the control parameters 310 for use by the control module 305 in affecting the selection, phase, timing and triggering the operation of the transducer elements 302. The control parameters 310 can be defined as trigger signals for triggering the generation and/or transmission of the modulated ultrasound signals via the transducer elements 302. Referring to FIG. 13A, one or more instructions may be stored on a memory 308 for affecting the operation of the transducer elements 302 in generating the ultrasound waves 311 and/or analyzing the echo signals 312.

In some embodiments, the annular ultrasound transducer arrays of the present invention are phased (e.g. as stored in the control parameters 310 for use by the control module 305), increasing speed and ease of use. Further, unlike a single transducer element probe, manual rotation is not required to generate a cross-sectional image when using the annular transducer of the present invention. Elimination of mechanical rotation reduces motion artefacts, enables faster imaging frame rate, and allows multiple focal spots in the ultrasound beams, which leads to an overall increase in image resolution (i.e. sharper image pixels).

Phased array systems pulse and receive signals from the plurality of elements of an array. The plurality of elements 302 is pulsed in a pattern to cause multiple beam components to combine with each other to form a single wave front 311 traveling in the desired direction. Similarly, the plurality of receiver elements 320 combine the echo input 312 into a single presentation. Because phasing technology permits electronic beam shaping and steering, it is possible to generate various ultrasonic beam profiles from a single probe assembly.

It is contemplated that in some embodiments (e.g. FIG. 13A), instructions stored in the memory 308 can be used by the control module 305 for execution by the processor 307 to control ultrasound beam angle, focal distance, and beam spot size (e.g. control parameters 310). These parameters can be dynamically scanned at each inspection point to optimize incident angle and signal-to-noise for each part geometry.

Referring to FIG. 13A, although the memory 308, processor 307, control module 305, control parameters 310, echo processor 309 have been shown as parts of the transducer array, in alternate embodiments, one or more of such components can be located on computing device 302 or on other computing devices coupled to (e.g. wirelessly) and in communication with the transducer array 301 (e.g. via the communication interface 306) and the transducer elements 302.

It is contemplated that in some embodiments, multiple-angle inspection can be performed with a single, small, multi-element probe and wedge, offering either single fixed angles or a scan through a range of angles. This method provides greater flexibility for inspection of complex geometries, such as cancellous bone. It is also contemplated that in some embodiments, multiplexing across many elements could allow motionless high-speed scans from a single transducer position. More than one scan may be performed from a single location with various inspection angles.

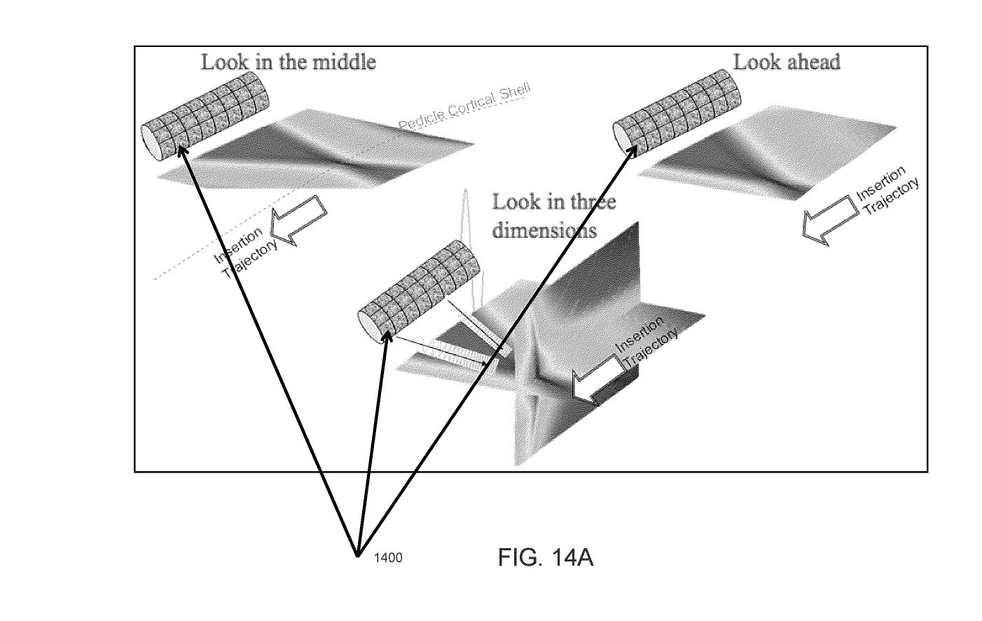

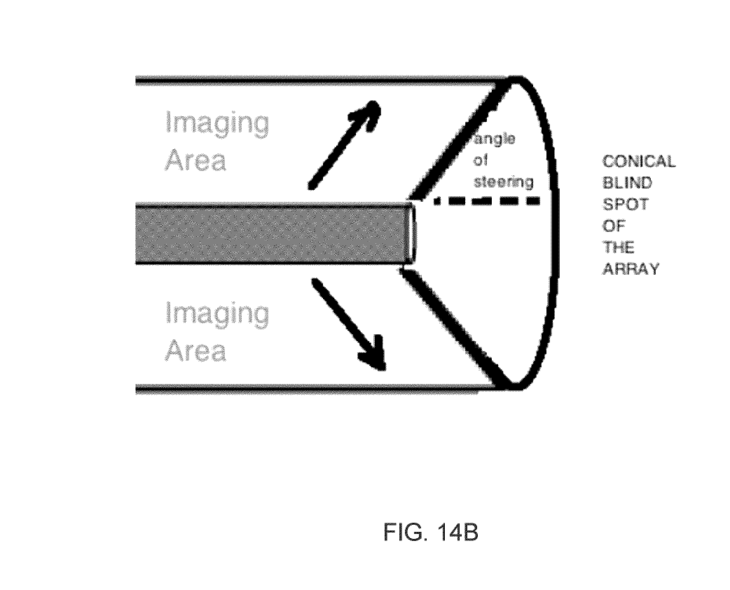

Referring to FIG. 14A, in some embodiments, the method further comprises transmitting modulated ultrasound signals directed forwardly relative to the insertional trajectory of the tool, wherein the forwardly directed ultrasound signals are transmitted from a plurality of the transducer elements 1400 and wherein the image produced is complimentary to a conical image, wherein the base of the cone is ahead of the tool along the insertional axis (FIG. 14B).

Referring to FIG. 15, in preferred embodiments, the imaged bone is a pedicle bone.

In preferred embodiments, the image is generated in real time. In one example, the transducer array is configured to process the echo signals received in real-time (e.g. via echo processor 309) and to communicate with an image processor (e.g. processor 316 of the computing device 302) to generate an image representative of the received echo signals on a display 315 of the user interface 313. An example image 1500 displayed on the display 315 is shown in FIG. 15.

In preferred embodiments, the image generated has an increased signal to noise ratio relative to an image generated from un-modulated ultrasound signals transmitted under identical conditions.

In some embodiments illustrated in FIG. 16A, the method for producing an image of bone using ultrasound further comprises noise reduction by signal (image) averaging. In this method, a plurality of modulated ultrasound signals is transmitted at the bone to be imaged at step 1600. The echoes of these modulated signals are received and averaged by the transducer array at steps 1602 and 1604.

In one embodiment, measuring the received plurality of echo signals at step 1602 further comprises demodulating the received echo signals corresponding to the modulated ultrasound signals.

Averaging reduces the random noise relative to the signal, thereby improving the signal to noise ratio. In another aspect, the averaging of the echo signals further comprises a weighted averaging, where particular echo signals are weighted higher as they are defined to be more relevant to the representative echo signal. For example, the echo processor 309 (e.g. shown in FIG. 13A) defines an optimal spatial positioning or focal point such that echo signals received within a range of the desired spatial positioning or focal point are provided with a higher weighting than the remaining received echo signals. Similarly, the echo processor 309 can be configured to measure the angles of each received echo signal and provide an increased weighting to particular received echo signals within a desired angle and/or positioning range for use in generating the representative echo signal (e.g. via a weighted averaging).

Transmitting a plurality of modulated ultrasound pulses (step 1600) and averaging the received echoes of the pulses (steps 1602, 1604) allows generation of a plurality of images from which an average can be taken, which minimizes the effect of random noise. In one aspect, the plurality of received echo signals are averaged to generate a representative echo signal 1604. The representative echo signal is transmitted for subsequent use in generating an averaged image (e.g. via image processor 316). In another aspect, an image is generated for each received echo signal and the images corresponding to each received echo signal are averaged after generation of the corresponding image (e.g. via the image processor 316). The process of averaging multiple images is preferable when the target is invariant, such as bone.

Referring now to FIG. 16, in some embodiments of the present invention, a block diagram depicting a system for ultrasound imaging of bone is provided, in accordance with one embodiment.

The system comprises a signal processor 1608 that codes or modulates at least one ultrasound signal. Various signal excitation coding methods that are known in the art can be used. In preferred embodiments, chirp modulation or golay code is used to modulate the transmitted signal(s). The signal processor 1608 also decodes at least one received echo signal of the at least one coded ultrasound signal.

The system also comprises an ultrasound transducer 1610 in communication with the signal processor 1608. The ultrasound transducer 1610 transmits the at least one coded ultrasound signal into the bone to be imaged. Preferably, the signal is transmitted at a low frequency, which is preferable in bone. The ultrasound transducer 1610 receives echoes of the coded ultrasound signal reflected from the bone to be imaged. The echoes of the coded ultrasound signal are communicated with the signal processor 1608 for performing demodulation thereof.

The system also comprises an image processor 1618 in communication with the signal processor 1608 (as shown in FIG. 13A, the image processor 316 may be present on an external computing device 302 and configured for receiving the demodulated echo signal from the transducer array 301, such as via the echo processor 309, for generating a representative image for display 315). In some embodiments, the system also comprises an image display 1616 in communication with the image processor 1618.

It is contemplated herein that the system for ultrasound imaging of bone described herein can comprise the annular ultrasound transducers described in U.S. Provisional Patent Application titled “Ultrasonic Array for Bone Sonography”, filed May 24, 2013, which names the inventors of the present application as inventors.

It is contemplated herein that the method and system for ultrasound imaging of bone described herein can be used in the method for predicting pedicle cortical breach described in U.S. Provisional Patent Application titled “Ultrasonic Array for Bone Sonography”, filed May 24, 2013, which names the inventors of the present application as inventors.

The invention will be more fully understood upon consideration of the following non-limiting examples.

### EXAMPLES

The present invention is further illustrated by the following examples, which should not be construed as limiting in any way.

### Example 1

### Comparing Methods of Coded-Excitation Signal Processing for Bone Sonography

In Example 1 coded excitation methods, in particular Chirp modulation and Golay codes (GC), were found to enhance quality of ultrasound images by increasing ultrasonic signal-to-noise ratios (SNR) while preserving resolution.

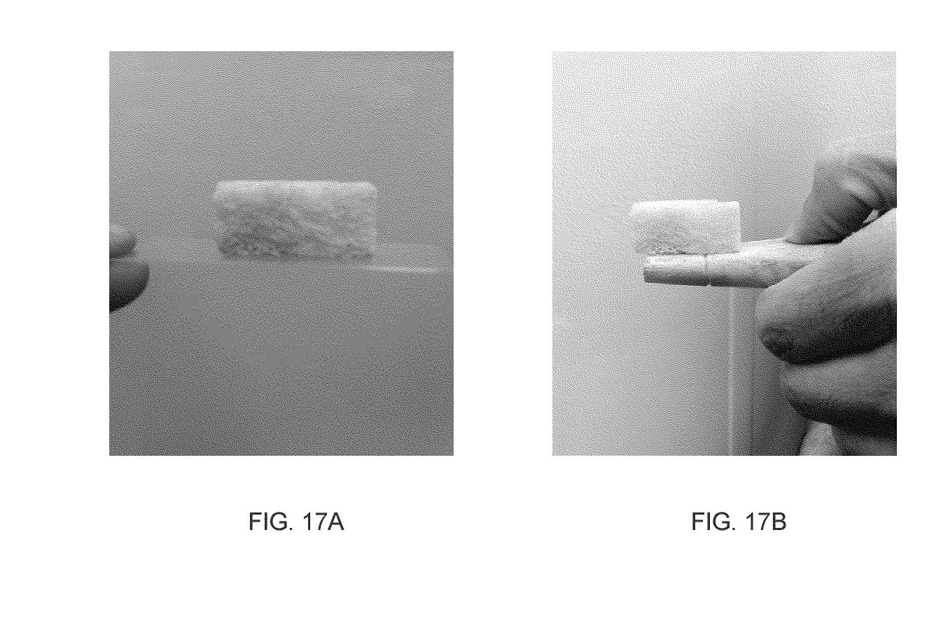

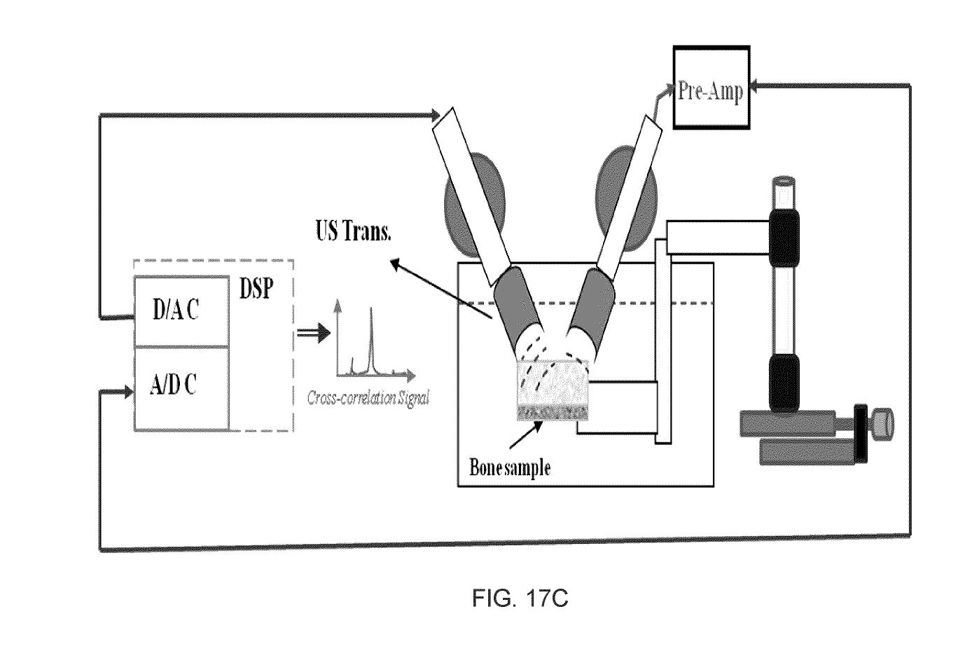

Three signal compression techniques were compared to one another by directing ultrasound pulses toward two substrates: i) a 1.5 cm thick human cancellous bone placed on top of a glass microscopic glass (FIG. 17A) and ii) a 1.5 cm thick human cancellous bone placed on top of a cortical bone layer (FIG. 17B). Pulse-echo experiments were performed on each substrate to determine whether detectable signals could be observed and distinguished from the background signal received from the cancellous bone. Experimental design is illustrated in FIG. 17C.

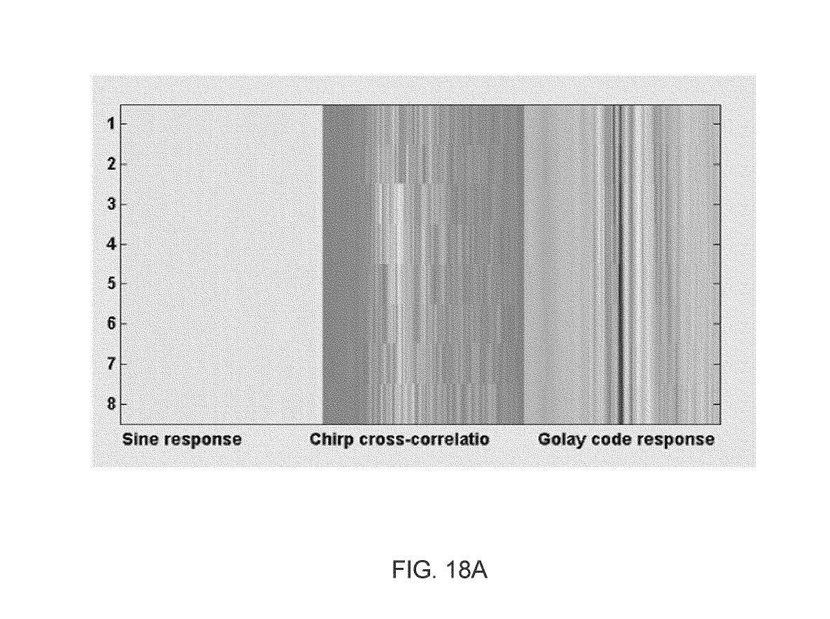

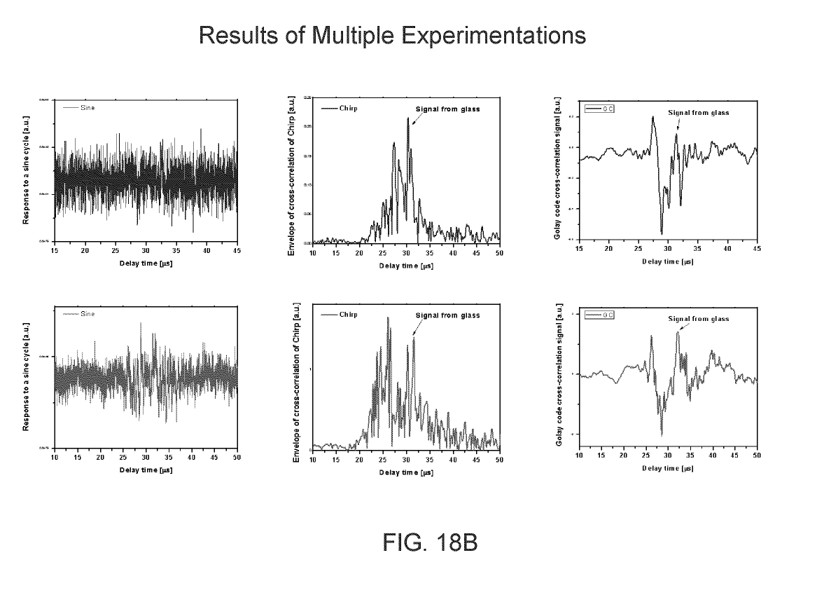

Referring to FIG. 18A-C, three signal compression techniques were compared: i) single sinusoidal pulse, ii) Chirp modulation, and iii) Golay Code. The input voltage in the pulse-echo setting was ±1V peak-to-peak, which served well for a proof-of-principle investigation. If the pulser and experimental setting allows, more voltage will improve sensitivity of the ultrasound imaging system. Sinusoidal excitation was not suitable to generate a strongly detectable signal (FIG. 8A-B, left-most images), whereas Chirp modulation and GC were both sufficient to generate detectable signals. The amplitude of detected signals was converted to a scaled color-map, which is illustrated by brightness-mode images for each excitation technique. The single sinusoidal pulse did not generate distinguishable characteristics in order to detect features such as the microscopic glass slice or the cortical layer.

### Example 2

### Enhancement of Signal to Noise Ratio in Modulated Ultrasound Signals

In Example 2, three SNR (Signal-to-Noise Ratio) enhancement methods were compared in simulation models based on using a 2 MHz transducer with 80% bandwidth.

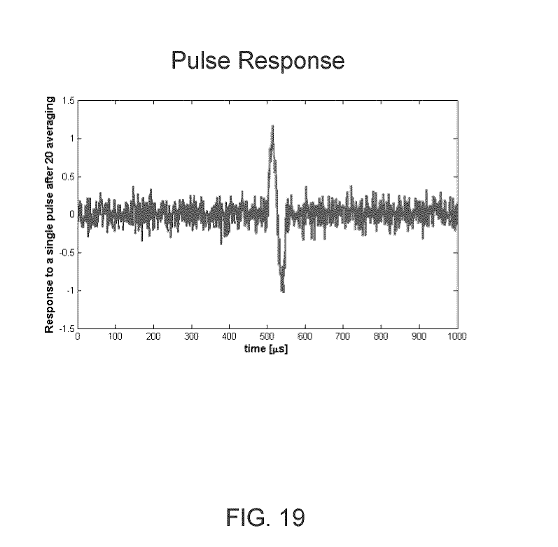

Referring to FIG. 19, the response of one cycle sine pulse (2MHz) after 20 times was averaged. The effect of averaging enhanced the SNR by square root of number of averages: {square root over ( N̅; where N̅ is the number of averaging. Square root function reaches a plateau relatively quickly, thus experimental work was required to determine a reasonable averaging outcome, without spending too much time taking more and more signals. Experimentation suggested that N=20-50 was a reasonable number.

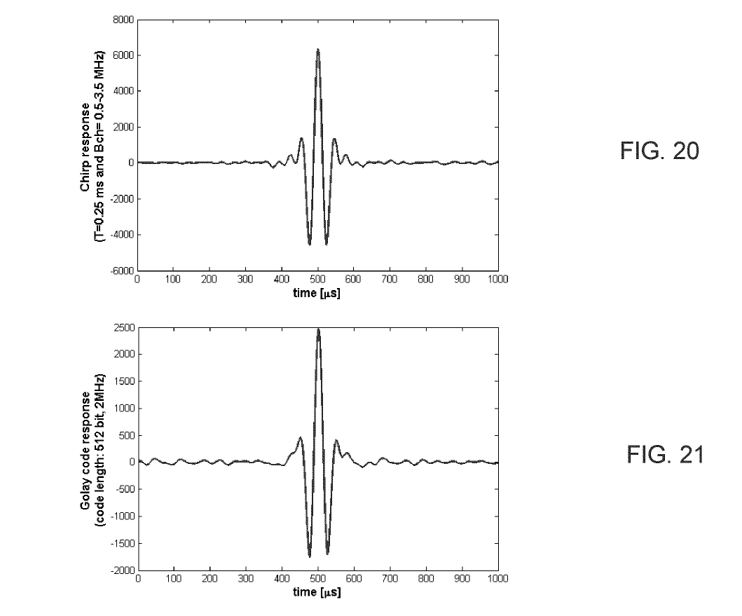

Referring to FIG. 20, the effect of chirp modulation employing a time duration of 0.25 ms (milliseconds) with a frequency sweeping from 0.5 to 3.5 MHz (Bandwidth of 3 MHz) was considered. Theoretically, the SNR enhancement was proportional to square root of time bandwidth product: \sqrt{(time)(Bandwidth)}=\sqrt{0.25(10³)0.3(10⁶)}=\sqrt{0.75(10³)}=\sqrt{750}≈30 times. The effect of averaging was also included, leading to higher SNR enhancement.

In experiments, a longer chirp duration of 1 [ms] was used which led into an enhancement of \sqrt{3000}≈55 times.

Referring to FIG. 21, the response of Golay code with L=512 bits modulated in 2 MHz was considered. Here the SNR enhancement was proportional to square root of double the code length: \sqrt{2L}\sqrt{2(512)}=\sqrt{1024}=32 times enhancement. Averaging was also included, leading to higher SNR enhancements.

The simulations in Example 2 are theoretical SNR enhancements that would occur under ideal conditions. Such conditions that might change when the effect of acoustic attenuation (damping) are present, such as in bone. However, the incorporation of averaging (for N times) will result in SNR enhancement of {square root over ( N̅ under conditions that are less than ideal.

The elements in the graphical user interfaces (GUIs), described herein are just for examples. There may be many variations to these GUI elements without departing from the spirit of the invention. For instance, buttons, images, graphs, and other GUI controls may be displayed and operated in a differing order, or buttons, images, graphs, and other GUI controls may be added, deleted, or modified.

The steps or operations in the flow charts described herein are just for examples. There may be many variations to these steps or operations without departing from the spirit of the invention. For instance, the steps may be performed in a differing order, or steps may be added, deleted, or modified.

Although the invention has been described with reference to certain specific embodiments, various modifications thereof will be apparent to those skilled in the art without departing from the purpose and scope of the invention as outlined in the claims appended hereto.

Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

We claim:

1. A computer implemented method for producing an image representative of a bone, the method comprising:
generating at least one modulated ultrasound signal, the at least one modulated signal being modulated in a range of 0.5 MHz to 5 MHz;
transmitting the at least one modulated ultrasound signal to the bone;
receiving a plurality of echo signals in response to the ultrasound transmission; and, demodulating each one of the plurality of echo signals and generating a representative echo signal from the plurality of echo signals;
wherein the representative echo signal is subsequently used for generating the image representative of the bone.

2. The method of claim 1, wherein generating the at least one modulated signal comprises modulating with a chirp frequency sweep.

3. The method of claim 2, wherein the chirp frequency sweep has a central frequency of 1 to 3 MHz and a bandwidth of one octave.

4. The method of claim 1, wherein generating the at least one modulated signal comprises modulating with Golay coding.

5. The method of any one of claims 1 to 4, wherein the at least one modulated ultrasound signal comprises a plurality of modulated ultrasound signals generated by an ultrasound transducer and transmitted outwardly by a plurality of transducer elements arranged in a ring configuration.

6. The method of claim 5, wherein the echoes are received by the plurality of transducer elements, wherein the plurality of transducer elements are in communication with at least one signal processor, wherein the at least one signal processor modulates and de-modulates the ultrasound, wherein the signal processor is in communication with an imaging processor, and wherein the image produced is a cross-sectional image.

7. The method of claim 1, wherein the image produced is a cross-sectional image.

8. The method of claim 5 or 6, further comprising generating ultrasound signals directed forwardly relative to an insertional trajectory of the tool, wherein the forwardly directed ultrasound signals are transmitted from the plurality of the transducer elements and wherein the image produced is complimentary to a conical image, wherein a base of the cone is ahead of the tool along the insertional axis.

9. The method of any one of claims 1 to 8, wherein the imaged bone is a pedicle bone.

10. The method of any one of claims 1 to 9, wherein the image is generated in real time.

11. The method of any one of claims 1 to 10, wherein generating a representative echo signal from the plurality of echo signals further comprises:
averaging the plurality of echo signals to reduce noise, the plurality of echo signals being averaged in dependence upon predefined criteria.

12. The method of claim 11 wherein the predefined criteria further comprises associating a weighting with each of the plurality of echo signals for generating the representative echo signal during a weighted averaging.

13. The method of claim 12, wherein the predefined criteria further comprises filtering at least one of the plurality of echo signals in dependence upon a spatial positioning associated with the echo signal prior to averaging.

14. A computer readable medium comprising a memory storing instructions for execution by one or more processors, the instructions when executed by said one or more processor implementing the methods of any one of claims 1 to 13.

15. A computer-implemented method for producing an image of bone using ultrasound, the method comprising:
a) acquiring ultrasound data by:
i) transmitting at least one modulated ultrasound signal at the bone to be imaged, wherein the signals are transmitted at frequencies in the range of 0.5 to 5 MHz, wherein the signals are reflected by features within the bone to produce echoes;
ii) measuring the echoes, wherein the measured echoes include echoes reflected from multiple spatial locations within the bone;
iii) demodulating the echoes; and
b) producing an image of the bone from the received demodulated echoes.

16. The method of claim 15, wherein the modulated signal comprises a chirp frequency sweep.

17. The method of claim 16, wherein the chirp frequency sweep has a central frequency of 2 to 3 MHz and a bandwidth of one octave.

18. The method of claim 17, wherein the modulated signal comprises Golay coding.

19. The method of any one of claim 15 to 18, wherein the at least one transmitted modulated ultrasound signal penetrates the bone to a depth of up to 2 cm.

20. The method of any one of claims 15 to 19, wherein the at least one transmitted modulated ultrasound signal penetrates the bone to a greater depth relative to an un-modulated ultrasound signal transmitted under identical conditions.

21. The method of any one of claims 15 to 20, wherein the at least one modulated ultrasound signal is a plurality of modulated ultrasound signals transmitted outwardly by a plurality of transducer elements arranged in a ring configuration, wherein the echoes are received by the plurality of transducer elements, wherein the plurality of transducer elements are in communication with at least one signal processor, wherein the at least one signal processor modulates and de-modulates the ultrasound, wherein the signal processor is in communication with an imaging processor, and wherein the image produced is a cross-sectional image.

22. The method of claim 21, wherein the outwardly directed modulated ultrasound signals are transmitted by a plurality of transducer elements arranged in a first plurality of ring configurations, wherein the echoes are received by a second plurality of ring configurations, wherein the first and second plurality of ring configurations are arranged adjacent to one another in a cylindrical configuration, each ring forming a row in the cylindrical configuration and wherein the image produced is a cylindrical image.

23. The method of claim 22, wherein the plurality of adjacent rings are mounted to or in or integrated with a tool and wherein the tool is inserted in the object to be imaged.

24. The method of claim 23, further comprising ultrasound signals directed forwardly relative to the insertional trajectory of the tool, wherein the forwardly directed ultrasound signals are transmitted from a plurality of the transducer elements and wherein the image produced is complimentary to a conical image, wherein the base of the cone is ahead of the tool along the insertional axis.

25. The method of any one of claims 15 to 24, wherein the imaged bone is a pedicle bone.

26. The method of any one of claims 15 to 25, wherein the image is generated in real time.

27. The method of any one of claims 15 to 26, wherein the image generated has an increased signal to noise ratio relative to an image generated from un-modulated ultrasound signals transmitted under identical conditions.

28. The method of any one of claims 15 to 27, further comprising:
noise reduction by signal (image) averaging, wherein a plurality of modulated ultrasound signals are transmitted at the bone to be imaged, one at a time, and the measured echoes are averaged.

29. A system for ultrasound imaging of bone, the system comprising
a) a signal processor, wherein the signal processor codes at least one ultrasound signal and wherein the signal processor decodes at least one received echo of the at least one coded ultrasound signal;
b) an ultrasound transducer in communication with the signal processor, wherein the ultrasound transducer transmits the at least one coded ultrasound signal into the bone to be imaged and wherein the ultrasound transducer receives echoes of the coded ultrasound signal reflected from the bone to be imaged;
c) a processor in communication with the signal processor for translating the at least one received echo to an image; and
d) a display in communication with the image processor for displaying the image.

30. A computer-implemented method for controlling an ultrasound transducer array, the method comprising:
i) communicating a predefined trigger signal to the ultrasound transducer array, the predefined trigger signal for defining at least one of: a selection of one or more transducer elements for transmitting a modulated ultrasound signal from the ultrasound transducer array, a directional angle of the transmitted ultrasound signal, time delays for transmitting the ultrasound signal;
wherein the communicated trigger signal is configured to control the ultrasound transducer array for at least one of: a generation and transmission of ultrasound signals therefrom.

31. The method of claim 30, further comprising providing predefined parameters for causing the ultrasound transducer array to:
generate the modulated ultrasound signal;
transmit the modulated ultrasound signal to the bone;
receive a plurality of echo signals in response to the ultrasound transmission; and, demodulate each one of the plurality of echo signals and generating a representative echo signal from the plurality of echo signals;
wherein the representative echo signal is subsequently used for generating the image representative of the bone.

32. A computer readable medium comprising a memory storing instructions for execution by one or more processors, the instructions when executed by said one or more processor implementing the method of claims 30 to 31.

33. A system for ultrasound imaging of a bone comprising:
an ultrasound transducer array comprising a plurality of transducer elements, a first set of the plurality of transducer elements configured for transmitting a modulated ultrasound signal and a second set of the plurality of transducer elements configured for receiving a plurality of echo signals in response thereto for subsequent generation of an image of the bone; and,
a processor in communication with the array, the processor comprising a memory and instructions stored thereon, the instructions when executed for generating control signals to control transmitting the modulated ultrasound signal in accordance with pre-defined parameters.

34. The system according to claim 33, wherein the parameters comprise: a selection of one or more transducer elements for transmitting a modulated ultrasound signal from the ultrasound transducer array, a directional angle of the transmitted ultrasound signal, a focus direction of the ultrasound transducer array, and time delays for transmitting the ultrasound signal.

35. The method according to claim 1, further comprising: generating the image on a display in dependence upon the representative echo signal.
